(19) United States
(12) Patent Application Publication
(10) Pub. No.: US 2026/0224840 A1
(43) Pub. Date: Aug. 6, 2026

(54) SYSTEM AND METHOD FOR WEARABLE AIRFLOW MODULATION AND RESPIRATORY REGULATION

(71) Applicant: Sharath K Kolachalam, Sugar Land, TX (US)

(72) Inventors: Sharath K. Kolachalam, Sugar Land, TX (US); Srinidhi Kolachalam, Sugar Land, TX (US)

(21) Appl. No.: 19/630,253

(22) Filed: Mar. 26, 2026

Related U.S. Application Data
(60) Provisional application No. 63/843,988, filed on Jul. 14, 2025.

(51) Int. Cl. A61M 21/02; A61M 39/24

(52) U.S. Cl. CPC A61M 21/02; A61M 39/24; A61M 2039/242; A61M 2039/244; A61M 2205/0216; A61M 2205/3331; A61M 2205/505; A61M 2209/088; A61M 2210/0606; A61M 2210/0618; A61M 2230/005; A61M 2230/42

(57) ABSTRACT

A wearable breathing system and method for airflow modulation and respiratory regulation are disclosed. The system comprises a wearable breathing system configured to be worn on a user's head or face, including a wearable support structure and a user-operable mode selection interface for selecting among a plurality of breathing exercise modes. Airflow actuation components provide mechanical, selective nasal airflow control in accordance with a selected mode, without requiring manual nostril occlusion by the user. The system supports asymmetric and ramp-controlled airflow modulation, enabling independent left and right nostril control and gradual application or release of airflow restriction. A hardware-implemented safety supervisor module enforces predefined physiological and mechanical safety limits independently of user commands, mobile applications, or cloud software. The system may further include redundant respiration sensing, fault detection with automatic reversion to a fully open airflow state, and calibration features for personalized operation.

### FIELD OF THE INVENTION

The present disclosure relates to wearable respiratory control systems, and more specifically to a system and method for mechanical airflow modulation and selective nasal airflow control for regulating breathing patterns.

### BACKGROUND OF THE INVENTION

Breathing techniques are commonly incorporated into health and wellness practices to support relaxation, focus, stress management, and regulation of physiological responses. Certain breathing methods involve intentional modulation of airflow patterns and timing across the breathing cycle, which may require coordinated control, sustained attention, and precise execution to achieve intended outcomes. In practice, maintaining consistency and accuracy during such breathing exercises can present challenges, particularly over extended durations or in environments where manual intervention is impractical.

Various approaches have been developed to support breathing-related activities, including instructional guidance, pacing cues, and physiological monitoring. Some approaches provide descriptive or audio-visual guidance intended to assist users in following breathing patterns, while others supply timing signals to indicate inhalation and exhalation intervals. Still other technologies focus on measuring respiratory or cardiovascular signals to provide feedback. While these approaches may offer partial assistance, they generally do not provide direct, physical modulation of airflow and rely on the user to manually execute or maintain the intended breathing pattern.

Manual control of breathing-related actions may introduce limitations related to user fatigue, posture constraints, cognitive load, or environmental compatibility. In certain contexts, such as extended use, multitasking scenarios, or immersive digital environments, reliance on continuous manual involvement may reduce usability or consistency. Additionally, existing solutions may not readily adapt to variations in user physiology, preferences, or contextual conditions, which can affect repeatability and overall user experience.

Further, prior breathing-support systems that provide guidance or monitoring typically do not incorporate active mechanical nostril occlusion combined with an independent, hardware-implemented safety enforcement mechanism configured to impose predefined physiological and mechanical limits irrespective of external software, mobile applications, or user commands. Systems lacking such hardware-level safeguards may rely primarily on software logic or user compliance and therefore may not provide fail-safe protection against excessive occlusion force, prolonged airflow restriction, or unintended actuation conditions.

Accordingly, there is an ongoing need for improved systems and methods that facilitate controlled breathing in a manner that is repeatable, adaptable, and compatible with hands-free operation. There is further a need for solutions capable of coordinating mechanical airflow modulation with timing guidance and control logic, while independently enforcing predefined safety constraints and accommodating different usage contexts and user needs. Addressing these considerations may enable more accessible, consistent, and safely regulated breathing support.

### SUMMARY

The following embodiments presents a simplified summary in order to provide a basic understanding of some aspects of the disclosed invention. This summary is not an extensive overview, and it is not intended to identify key/critical elements or to delineate the scope thereof. Its sole purpose is to present some concepts in a simplified form as a prelude to the more detailed description that is presented later.

The present disclosure provides an integrated wearable breathing framework that unifies airflow modulation, user interaction, physiological safety enforcement, and adaptive control within a coordinated, system-level architecture. Conventional breathing-assistance approaches typically rely on instructional guidance, timing cues, or passive monitoring, thereby placing the burden of execution on the user and limiting consistency and hands-free operation. In contrast, the framework described herein employs wearable airflow actuation, configurable breathing modes, embedded safety supervision, and adaptive calibration mechanisms to enable guided breathing exercises with improved usability, repeatability, and safety. In certain embodiments, system operation may be governed by defined operational states, including an authorized training state in which airflow modulation is permitted in accordance with a selected breathing exercise mode, and a non-training state in which airflow pathways remain fully open and active modulation is inhibited.

According to some example embodiments, the present disclosure provides a method of operating a wearable breathing system worn on a user's head or face. The method includes receiving user input via a user-operable mode selection interface, selecting a breathing exercise mode from among a plurality of predefined modes, and controlling airflow associated with at least one nostril in accordance with the selected mode without requiring manual nostril occlusion by the user. The method may further include enforcing predefined physiological and mechanical safety limits using a hardware-implemented safety supervisor module, automatically reverting the system to a fully open airflow state in response to abnormal conditions, and applying gradual airflow transitions to prevent abrupt physiological effects. In some embodiments, the hardware-implemented safety supervisor module may be realized as a physically separate microcontroller, watchdog circuit, field-programmable gate array (FPGA), dedicated comparator circuitry, or analog threshold logic configured to operate independently of primary control software.

According to some example embodiments, the present disclosure provides a wearable breathing system comprising a wearable support structure, a mode selection interface configured to receive mechanical and/or non-mechanical user input, airflow actuation components, and a hardware-implemented safety supervisor module operating independently of external software. The system may further include redundant respiration sensing elements with fault detection, passive mechanical airflow bypass paths, personalization and calibration modules, self-aligning nostril interfaces, asymmetric nostril control profiles, modular actuator interfaces, fit-detection sensors, and usage tracking for disposable nasal-contact elements. In certain embodiments, the passive mechanical airflow bypass path may include a spring-biased flap valve, pressure-relief diaphragm, or comparable mechanically responsive element configured to automatically open when airflow resistance exceeds a predefined threshold, independent of electronic actuation or power availability. These components cooperatively manage airflow modulation, ensure proper fit and alignment, and maintain safe operation across varying usage conditions.

According to some example embodiments, the present disclosure provides one or more non-transitory computer-readable media storing instructions that, when executed by processing circuitry of the wearable breathing system, cause the system to perform breathing-mode selection, airflow control, safety enforcement, calibration, fault handling, and session evaluation operations described herein. The instructions may further cause calculation and presentation of session effectiveness metrics and adaptive adjustment of breathing parameters over time.

According to some example embodiments, taken together, the systems and methods of the present disclosure provide a comprehensive wearable breathing architecture that integrates mechanical airflow control, embedded safety supervision, adaptive calibration, and user interaction to enable hands-free, guided breathing exercises across a range of wellness and usage contexts.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

The above and still further example embodiments of the present invention will become apparent upon consideration of the following detailed description of embodiments thereof, especially when taken in conjunction with the accompanying drawings, and wherein:

FIG. 1 is an exploded view of a wearable breathing system, in accordance with an example embodiment.

FIG. 2 is an electrical schematic and functional control architecture of a wearable breathing system, in accordance with an example embodiment.

FIG. 3 illustrates a block diagram of a wearable breathing system showing interaction between system components, in accordance with an example embodiment.

FIG. 4 is a block diagram of an exemplary workflow wearable breathing system, in accordance with an example embodiment.

FIG. 5 illustrates a flow diagram of a wearable breathing system to perform a method of operating a wearable breathing system worn on a user's head or face, in accordance with an example embodiment.

The figures illustrate embodiments of the invention for purposes of illustration only. One skilled in the art will readily recognize from the following description that alternative embodiments of the structures and methods illustrated herein may be employed without departing from the principles of the invention described herein.

### DETAILED DESCRIPTION

In the following description, for purposes of explanation, numerous specific details are set forth to provide a thorough understanding of the present invention. It will be apparent, however, to one skilled in the art that the present invention can be practiced without these specific details. In other instances, systems, apparatuses, and methods are shown in block diagram form only to avoid obscuring the present invention.

Reference in this specification to “one embodiment” or “an embodiment” or “example embodiment” means that a particular feature, structure, or characteristic described in connection with the embodiment is included in at least one embodiment of the present invention. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity but rather denote the presence of at least one of the referenced items. Moreover, various features are described which may be exhibited by some embodiments and not by others. Similarly, various requirements are described which may be requirements for some embodiments but not for other embodiments.

Some embodiments of the present disclosure will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout.

The terms “comprise”, “comprising”, “includes”, or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus proceeded by “comprises... a” does not, without more constraints, preclude the existence of other elements or additional elements in the system or method.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present invention. Further, it is to be understood that the phraseology and terminology employed herein are for the purpose of the description and should not be regarded as limiting. Any heading utilized within this description is for convenience only and has no legal or limiting effect.

### Definitions

The term “fully open airflow state” refers to an operational condition of the wearable breathing system in which airflow through one or both nostrils is substantially unrestricted by active actuation components of the system. In this state, any controllable occlusion mechanisms are positioned or configured so as not to intentionally impede airflow beyond inherent structural presence, thereby allowing natural respiration without imposed airflow modulation. The fully open airflow state may be entered automatically in response to safety triggers, fault detection, power interruption, or user command.

The term “training interval” refers to a defined temporal segment during which the wearable breathing system actively applies airflow modulation in accordance with a selected breathing exercise mode. A training interval may correspond to inhalation, exhalation, breath retention, or a composite phase within a breathing cycle, and may include predefined duration, ramp characteristics, and occlusion parameters.

The term “passive mechanical airflow bypass path” refers to a mechanically responsive airflow pathway configured to permit airflow independently of electronic control, software commands, or powered actuation. The bypass path is structured to automatically open or increase airflow when a predefined pressure, resistance, or force threshold is exceeded, thereby providing a fail-safe airflow route under abnormal or unpowered conditions.

The term “compliant” may be used to refer to a structure, material, or component that is capable of elastically deforming, flexing, yielding, or otherwise conforming in response to an applied force, pressure, or contact condition, and substantially returning toward its original shape upon removal of the applied force.

The term “actuation” refers to the controlled initiation, modulation, or termination of mechanical movement, force application, displacement, or positional adjustment of a component in response to an electrical, mechanical, pneumatic, magnetic, or other control input.

The term “acoustic sensors” may be used to refer to devices configured to detect, measure, or monitor sound waves, pressure fluctuations, or vibrational energy transmitted through air, solid structures, or body tissues, and to generate corresponding electrical signals representative of such detected acoustic phenomena.

### End of Definitions

The present disclosure provides a wearable breathing system configured to facilitate nostril breathing through controlled airflow modulation without requiring manual nostril occlusion by the user. The system is designed to be worn on a user's head or face and to selectively regulate airflow associated with at least one nostril in accordance with a selected breathing exercise mode. By integrating mechanical airflow actuation, physiological sensing, safety enforcement, calibration, and user-selectable breathing programs within a single coordinated architecture, the wearable breathing system enables structured, repeatable, and hands-free execution of nostril-based breathing exercises.

In certain embodiments, the wearable breathing system comprises a wearable support structure and a user-operable mode selection interface coupled to the wearable support structure. The wearable support structure is configured to be worn on a user's head or face and may take the form of eyewear, a head-mounted assembly, a helmet-style apparatus, or a structure integrated with augmented or virtual reality headsets. The wearable support structure maintains stable positioning relative to the nasal region such that airflow actuation components remain properly aligned with the user's nostrils during stationary use and movement.

The user-operable mode selection interface enables the user to select between a plurality of breathing exercise modes for facilitating nostril breathing. In certain embodiments, the mode selection interface comprises at least one manually actuated input device configured to receive mechanical user input, such as a button, slider, or switch. In other embodiments, the mode selection interface includes at least one input device configured to receive user input via one or more non-mechanical input modalities, including touch, tap, gesture, or voice. Regardless of modality, the mode selection interface allows the user to initiate, configure, and control structured breathing sessions directly from the wearable breathing system.

Upon receiving user input via the user-operable mode selection interface, the wearable breathing system selects a breathing exercise mode and controls airflow associated with at least one nostril in accordance with the selected mode. Importantly, the method of operation is performed without requiring manual nostril occlusion by the user, thereby eliminating hand involvement and improving repeatability and usability.

Central to the wearable breathing system are airflow actuation components configured to selectively restrict airflow through one or both nostrils. In one embodiment, the airflow actuation components include compliant nasal-contact elements positioned adjacent to the nostrils. These elements may be driven by electromechanical actuators that translate rotational or linear motion into controlled nostril occlusion. The compliance of the nasal-contact elements allows adaptation to varying nasal geometries while minimizing discomfort.

The airflow actuation components are capable of independent control of left and right nostrils, enabling alternate-nostril breathing sequences and other structured airflow patterns. In certain embodiments, the system supports asymmetric nostril control profiles in which occlusion timing, occlusion force, and airflow resistance differ independently between the left and right nostrils. This asymmetric capability allows the wearable breathing system to implement advanced breathing protocols and individualized control strategies.

To prevent abrupt physiological transitions, the wearable breathing system may enforce gradual ramp-up and ramp-down phases when applying or releasing airflow restriction. Such ramping may be implemented through controlled actuation trajectories that progressively increase or decrease occlusion force or airflow resistance over a defined time interval. In some embodiments, ramp control is implemented by pulse-width modulation of a motor driver, controlled displacement of a linear actuator, servo position interpolation, or closed-loop force control based on sensed pressure feedback.

To ensure safe operation, the wearable breathing system may further comprise a hardware-implemented safety supervisor module operatively coupled to the airflow actuation components. The hardware-implemented safety supervisor module enforces predefined physiological and mechanical safety limits independently of user commands, mobile applications, or cloud software. This independence ensures that safety constraints cannot be overridden by external systems or software layers.

In certain structural embodiments, the safety supervisor module is implemented as a physically separate hardware element from a primary control processor. For example, the safety supervisor module may comprise: a dedicated microcontroller separate from a primary system processor and configured to independently validate actuation commands before drive signals are transmitted to the airflow actuation assembly; a hardware watchdog circuit configured to monitor timing intervals and force thresholds and to disable actuator drive signals upon detection of abnormal conditions; a field-programmable gate array (FPGA) configured to implement state-based gating logic that conditionally permits or blocks actuator control signals; or analog comparator logic configured to directly compare sensed force, pressure, or airflow signals against predefined threshold voltages and to physically interrupt actuator drive circuitry when a threshold is exceeded.

In some embodiments, the safety supervisor module is electrically interposed in a command path between the control processor and a motor driver circuit, such that actuator drive signals must pass through the safety supervisor before reaching the airflow actuation assembly. In such configurations, the safety supervisor module may include hardware gating transistors, relays, or driver-enable lines configured to physically inhibit actuator energization upon detection of a safety violation

The safety supervisor module may monitor at least one of a maximum nostril occlusion force, a maximum continuous nostril closure duration, a minimum airflow allowance, a minimum respiratory rate, a maximum respiratory rate, or a maximum breath-retention duration. If an actuation command would violate any predefined safety limit, the hardware-implemented safety supervisor module overrides the command and may automatically revert the system to a fully open airflow state by disabling motor drive power or commanding release of the occlusion mechanism.

The safety framework may also include a passive mechanical airflow bypass path configured to automatically open when airflow resistance exceeds a predefined threshold. Because the passive mechanical airflow bypass path operates independently of electronic actuation or power availability, it provides an additional fail-safe airflow mechanism in the event of power loss or actuator malfunction.

In certain embodiments, the passive mechanical airflow bypass path comprises: a spring-biased flap valve positioned within an airflow channel and configured to open when differential pressure across the flap exceeds a predetermined spring force threshold; a pressure-relief diaphragm formed from an elastomeric membrane configured to deform and create an auxiliary airflow aperture when internal nasal pressure exceeds a predefined limit; a pressure-actuated sliding gate mechanically biased toward an open position in the absence of actuator force; or a mechanically latched occlusion element configured to default to an open state upon loss of electrical power.

These passive elements may be integrated within the nostril interface housing or positioned in parallel airflow conduits such that unrestricted airflow is mechanically restored when pressure exceeds a safe threshold, independent of electronic control signals.

In certain embodiments, the wearable breathing system includes redundant or heterogeneous respiration sensing elements configured to detect airflow, respiratory timing, or related physiological parameters. These respiration sensing elements may include airflow sensors, pressure sensors, acoustic sensors, or other modalities capable of characterizing breathing behaviour.

A fault-detection engine may process signals from the respiration sensing elements to detect sensor malfunction, misalignment, or displacement. The fault-detection engine may be implemented in hardware, firmware, or a combination thereof. When sensing inconsistency is detected, the wearable breathing system may automatically revert to a fully open airflow state by disabling actuator drive signals and/or releasing the occlusion mechanism.

In some embodiments, sensing data may also be used to validate correct execution of the selected breathing exercise mode, to adjust ramp profiles dynamically, and to compute session metrics representing breathing consistency, adherence to timing intervals, or airflow stability.

To accommodate anatomical variability among users, the wearable breathing system may include a personalization and calibration module configured to perform an automatic nostril calibration routine. During calibration, the system establishes an individualized minimum effective occlusion force and actuator geometry based on nasal compliance and airflow response. Calibration may be performed prior to initiating a breathing exercise mode or periodically to maintain performance consistency.

In one exemplary embodiment, the calibration module executes a controlled incremental force application sequence. The nostril actuation assembly applies a first low occlusion force to a selected nostril while respiration sensing elements sample airflow and/or pressure characteristics. The applied force is incrementally increased in predefined force steps or displacement increments, and airflow is sampled at each increment. The system evaluates airflow reduction relative to baseline airflow and determines whether a predefined occlusion effectiveness threshold has been reached. A convergence criterion may be defined as a percentage reduction in airflow, stabilization of measured airflow over multiple sampling intervals, or achievement of a target resistance value. Once the convergence threshold is satisfied, the system records the corresponding actuator position and force as the individualized minimum effective occlusion force. The calibration routine may also determine nasal compliance characteristics by analyzing force-versus-airflow response curves and may store individualized parameters for subsequent actuation control. The calibration process may be repeated independently for left and right nostrils.

In conjunction with calibration, the wearable device may include a self-aligning nostril interface configured to dynamically adjust actuator positioning to maintain consistent alignment during user movement or extended wear. This self-alignment feature ensures that the airflow actuation components remain properly positioned relative to the nostrils even as the wearable support structure experiences minor shifts. Fit-detection sensors positioned near a nasal-contact region may assess contact quality and alignment before initiating a breathing session. The wearable breathing system may verify nasal-contact fit prior to actuation and provide corrective guidance if misalignment is detected.

In certain embodiments, the wearable device includes a modular actuator interface having standardized mechanical and electrical connectors configured to interchangeably receive multiple nostril-actuation modules. Each nostril-actuation module may be automatically identified upon installation so that corresponding control limits and calibration parameters are applied. The wearable breathing system may further comprise disposable or limited-use nasal-contact elements tracked by a usage counter. The usage counter may prompt replacement after a predetermined usage threshold to maintain hygiene and consistent mechanical properties.

During operation, the wearable breathing system executes the selected breathing exercise mode by controlling airflow through at least one nostril in accordance with programmed timing and sequencing. The method may further comprise enforcing safety limits using the hardware-implemented safety supervisor module and automatically reverting to a fully open airflow state if airflow resistance exceeds a predefined threshold or if sensing inconsistency is detected. Upon completion of a breathing session, the system may calculate at least one session metric score and present the score as an indication of breathing session effectiveness. Session metrics may reflect adherence to timing, consistency of nostril alternation, compliance with selected breathing parameters, or other performance indicators derived from respiration sensing elements.

In certain embodiments, the hardware-implemented safety supervisor module is implemented as a physically separate hardware component from a primary control processor. For example, the safety supervisor module may comprise a dedicated secondary microcontroller configured to independently validate actuation commands prior to energizing actuator drivers. In other embodiments, the safety supervisor module may comprise a watchdog circuit configured to monitor occlusion duration and actuator activity timing and to disable actuator drive signals upon detection of abnormal operation. In further embodiments, the safety supervisor module may be implemented using field-programmable gate array (FPGA) logic configured to perform deterministic state-based gating of actuator control signals. In still other embodiments, the safety supervisor module may include analog comparator circuitry configured to compare sensed pressure, airflow, or force signals against predefined voltage thresholds and to physically interrupt actuator drive lines when a threshold is exceeded. In certain configurations, the safety supervisor module is electrically interposed between the primary control processor and the motor driver circuitry such that actuator drive enable signals must pass through the safety supervisor prior to reaching the airflow actuation assembly.

In certain embodiments, the wearable breathing system operates within a distributed architecture in which real-time airflow control and safety enforcement remain resident on the wearable device. A mobile computing device may provide user interface functionality, session configuration, visualization of session metric scores, and non-safety-critical personalization processing. Optional cloud-based services may support long-term storage, analytics, or update distribution. However, the hardware-implemented safety supervisor module and airflow actuation components remain locally controlled such that safety enforcement is independent of user commands, mobile applications, or cloud software. In such embodiments, the safety supervisor may be implemented as a physically separate microcontroller, FPGA-based gating module, watchdog timing circuit, or analog comparator network integrated on the wearable device's printed circuit board and electrically positioned in the actuator command path.

The wearable breathing system differentiates between periods during which airflow actuation is authorized and periods during which actuation is disabled. Actuation may be enabled only after user input via the user-operable mode selection interface and verification of fit, sensor integrity, and system readiness. Outside active breathing sessions, airflow actuation components may be maintained in a fully open state, and actuation commands may be rejected to prevent unintended airflow restriction. In certain embodiments, passive monitoring of respiration sensing elements continues outside active sessions to detect abnormal airflow obstruction or improper device positioning; however, actuator drive circuitry remains electrically disabled through hardware gating logic until explicit user initiation is confirmed. Accidental activation may be prevented through dual-confirmation mechanisms, such as requiring both a user input confirmation and a verified fit-detection signal before enabling actuator drive power. In some embodiments, actuator enable lines are controlled by both the primary control processor and the hardware safety supervisor module in a dual-channel gating configuration, such that both channels must assert an enable condition before actuation is permitted.

If at any time predefined safety limits are reached, airflow resistance exceeds a predefined threshold, or sensor malfunction is detected, the system automatically transitions to a fully open airflow state and may require re-initiation through the user-operable mode selection interface before resuming operation.

Taken together, the wearable breathing system provides an integrated mechanical, electronic, and safety-controlled platform for facilitating nostril breathing in a hands-free manner. By combining user-selectable breathing exercise modes, controlled airflow actuation, independent hardware safety supervision, redundant respiration sensing, automatic calibration, modular components, and session performance metrics within a single wearable support structure, the present disclosure enables structured, safe, and repeatable nostril-based breathing practice across diverse usage contexts.

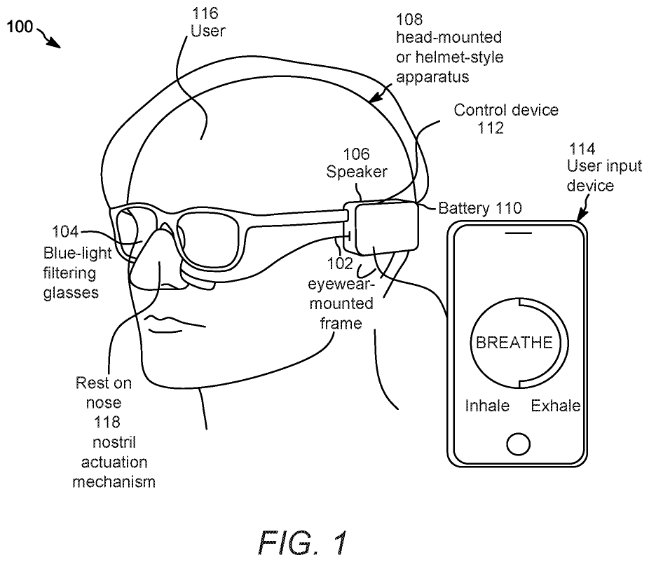

Embodiments of the present disclosure may provide a system and a method of operating a wearable breathing system worn on a user's head or face. The system and the method of operating a wearable breathing system worn on a user's head or face are described with reference to FIG. 1 to FIG. 5 as detailed below.

FIG. 1 illustrates an example embodiment of a wearable breathing system 100 that integrates structural support, nostril airflow control, power supply, user feedback, and external device communication within a head-mounted configuration. As shown, the wearable breathing system 100 is configured to be worn on a head of a user 116 and to facilitate guided nostril breathing exercises through controlled airflow modulation.

The wearable breathing system 100 includes a head-mounted or helmet-style apparatus 108 configured to be supported on the head of the user 116. In the illustrated embodiment, the head-mounted or helmet-style apparatus 108 is integrated with or supported by an eyewear-mounted frame 102. The eyewear-mounted frame 102 may be configured similarly to conventional eyeglasses and may rest on the user's nose and ears. The eyewear-mounted frame 102 may include blue-light filtering glasses 104. While illustrated as blue-light filtering glasses, the lenses 104 may alternatively or additionally include corrective lenses, protective lenses, or non-optical elements.

A nostril actuation mechanism 118 is positioned adjacent to the nasal region of the user 116. The nostril actuation mechanism 118 is configured to selectively restrict airflow through at least one nostril of the user. In certain embodiments, the nostril actuation mechanism 118 may include compliant or flexible elements configured to move between an open state and a restricted airflow state. The nostril actuation mechanism 118 may be supported by or coupled to the eyewear-mounted frame 102.

The wearable breathing system 100 further includes a control device 112 mounted to or integrated within the head-mounted or helmet-style apparatus 108. The control device 112 may include one or more processors, control circuitry, memory, and communication components configured to control operation of the nostril actuation mechanism 118 and other system components.

A battery 110 is electrically coupled to the control device 112 and provides electrical power to the wearable breathing system 100. The battery 110 may be rechargeable and may be integrated within the head-mounted or helmet-style apparatus 108. A speaker 106 is also coupled to the head-mounted or helmet-style apparatus 108. The speaker 106 may be configured to provide audible guidance, breathing cues, alerts, or feedback to the user 116 during operation of the system.

In certain embodiments, the wearable breathing system 100 may communicate with an external user input device 114. The user input device 114 may comprise a mobile device, such as a smartphone, tablet, or similar computing device. The user input device 114 may execute an application configured to allow a user to select breathing modes, configure session parameters, receive feedback, and monitor session metrics. Communication between the control device 112 and the user input device 114 may occur via wired or wireless communication protocols.

During operation, the user 116 wears the head-mounted or helmet-style apparatus 108 such that the nostril actuation mechanism 118 is positioned adjacent to the nostrils. The control device 112 controls the nostril actuation mechanism 118 in accordance with selected breathing modes, which may be selected via the wearable system 100 itself or via the user input device 114. Audible guidance may be delivered through the speaker 106.

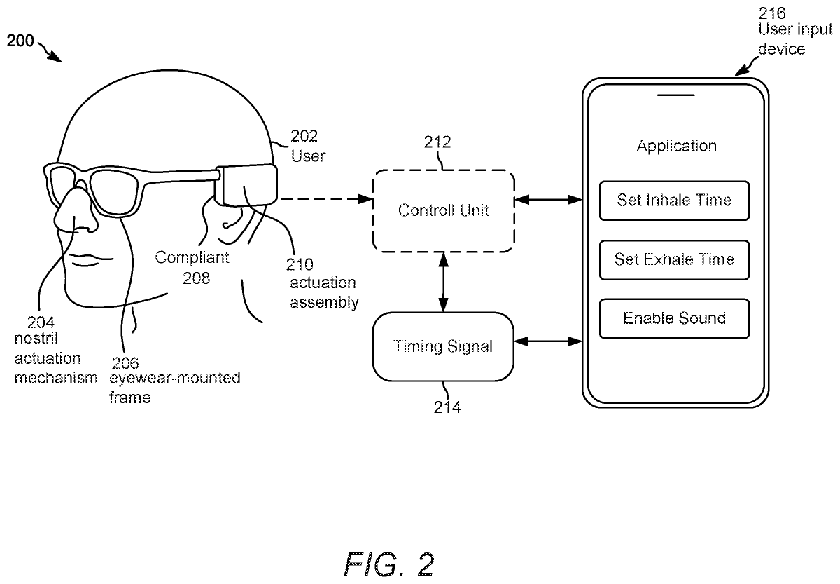

FIG. 2 illustrates an example embodiment of the electrical control and motor drive architecture for the wearable breathing system 200, showing the interaction between the wearable hardware components and an external user input device 216. As shown, the wearable breathing system 200 is configured to be worn by a user 202 and to provide controlled nostril airflow modulation through an electronically controlled actuation assembly.

The wearable breathing system 200 includes an eyewear-mounted frame 206 configured to be supported on the head of the user 202. The eyewear-mounted frame 206 supports a nostril actuation mechanism 204 positioned adjacent to the nasal region of the user 202. The nostril actuation mechanism 204 may include compliant elements configured to selectively restrict airflow through at least one nostril.

In the illustrated embodiment, the nostril actuation mechanism 204 is operatively coupled to an actuation assembly 210. The actuation assembly 210 may include a motor, actuator, or other powered drive system configured to move the nostril actuation mechanism 204 between open and restricted airflow states. A compliant interface region 208 is shown positioned between the eyewear-mounted frame 206 and the nasal region of the user 202. The compliant interface 208 may include flexible or elastomeric materials configured to conform to the user's facial geometry and enhance comfort and sealing performance.

The actuation assembly 210 is electrically coupled to a control unit 212. The control unit 212 may include one or more processors, microcontrollers, driver circuitry, and associated memory configured to generate control signals for operating the actuation assembly 210. The control unit 212 is further operatively coupled to a timing signals module 214. The timing signals module 214 may generate inhale and exhale timing intervals, phase transitions, or duty cycles used to coordinate nostril occlusion patterns. In some embodiments, the timing signals module 214 may be implemented in software, firmware, hardware, or a combination thereof within or associated with the control unit 212.

The wearable breathing system 200 may communicate with a user input device 216, such as a smartphone or other mobile computing device. The user input device 216 may execute an application that allows a user to configure breathing parameters and operational modes. As illustrated, the application running on the user input device 216 may provide user-selectable controls including: set inhale time, set exhale time, enable sound. The user input device 216 may communicate with the control unit 212 via wired or wireless communication protocols, such as Bluetooth®, Wi-Fi®, or other suitable communication technologies. Communication between the control unit 212 and the user input device 216 may be bidirectional, enabling configuration commands to be transmitted to the control unit 212 and operational or status data to be transmitted back to the user input device 216.

During operation, the user 202 wears the eyewear-mounted frame 206 such that the nostril actuation mechanism 204 is positioned adjacent to the nostrils. The control unit 212 receives configuration inputs from the user input device 216 and generates timing signal 214 corresponding to selected inhale and exhale durations. The control unit 212 drives the actuation assembly 210 in accordance with the generated timing signal 214, thereby selectively actuating the nostril actuation mechanism 204 to control airflow through the user's nostrils. Audible guidance or sound cues may be enabled through application settings, and such cues may be synchronized with the timing signal 214.

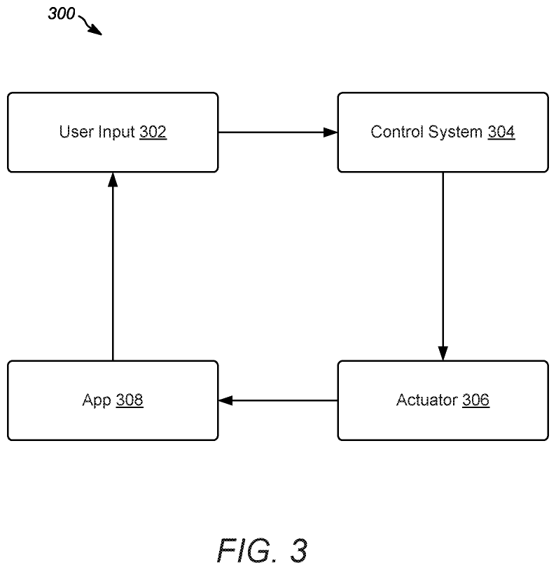

FIG. 3 illustrates the functional interaction among user input, control processing, actuation, and application-level interface components of the wearable breathing system 300. As shown, the wearable breathing system 300 includes a user input module 302, a control system 304, an actuator 306, and an application module 308. The blocks represent functional components that may be implemented in hardware, software, firmware, or combinations thereof.

The user input module 302 is configured to receive input from a user. In various embodiments, the user input module 302 may include one or more manually actuated input devices (e.g., buttons, switches, knobs), touch-sensitive elements, gesture sensors, voice input interfaces, or inputs received via an external mobile device application. The user input module 302 generates input signals corresponding to user-selected breathing modes, timing parameters, or other operational settings. The user input module 302 provides input signals to the control system 304, as indicated by the directional arrow.

The control system 304 is configured to process input signals received from the user input module 302 and to generate control commands for regulating nostril airflow. The control system 304 may include one or more processors, microcontrollers, memory devices, timing circuits, and driver circuitry. The control system 304 may execute breathing protocols defining inhale duration, exhale duration, nostril switching intervals, or other airflow control parameters. The control system 304 outputs control signals to the actuator 306.

The actuator 306 represents an actuation mechanism configured to physically modulate airflow associated with at least one nostril of a user. In some embodiments, the actuator 306 may comprise a motor, linear actuator, servo mechanism, solenoid, or other powered drive system operatively coupled to a nostril actuation mechanism. Upon receiving control signals from the control system 304, the actuator 306 transitions between open and restricted airflow states in accordance with the selected breathing mode. The actuator 306 may also provide feedback or status information to the application module 308, as indicated by the directional arrow from the actuator 306 to the application module 308.

The application module 308 (App 308) may be implemented on a mobile device, wearable companion device, or onboard processor. The application module 308 may provide a graphical user interface for configuring breathing parameters, displaying session information, and monitoring device status. As shown, the application module 308 communicates with the user input module 302. This interaction may represent user interaction through the application interface or synchronization between the wearable system and an external device. The communication path may be wired or wireless.

In operation, user input received at the user input module 302 is transmitted to the control system 304. The control system 304 processes the input and generates control commands for the actuator 306. The actuator 306 modulates nostril airflow accordingly. Operational data and status information may be communicated to the application module 308, which may present feedback to the user and optionally transmit updated configuration parameters back to the user input module 302.

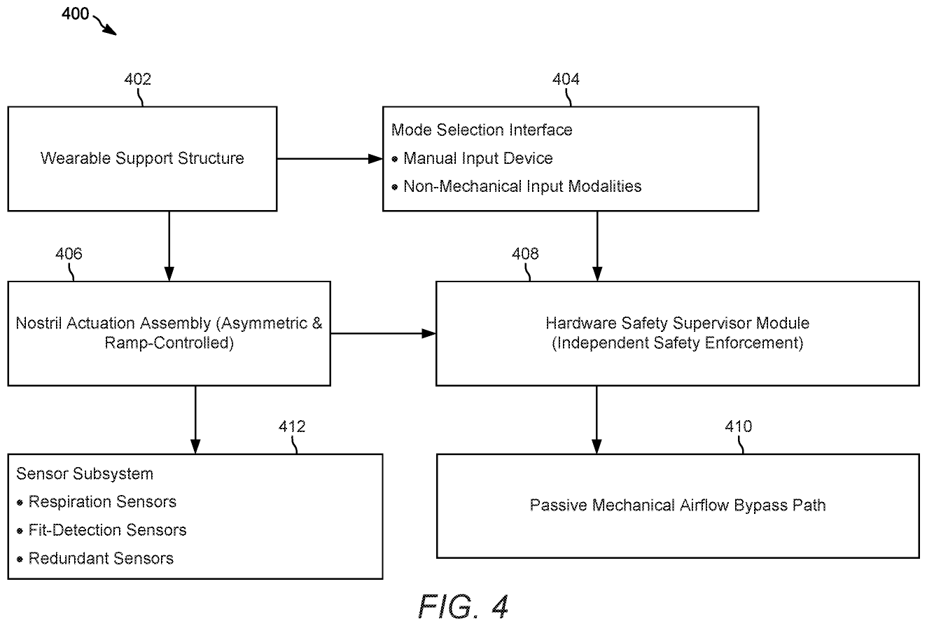

FIG. 4 illustrates an exemplary workflow and safety-oriented architecture of the wearable breathing system 400, showing the interaction among user input, actuation control, sensing, and independent safety mechanisms. As shown, the wearable breathing system 400 includes a wearable support structure 402, a mode selection interface 404, a nostril actuation assembly (Asymmetric & Ramp-controlled) 406, a hardware safety supervisor module (independent safety Enforcement) 408, a passive mechanical airflow bypass path 410, and a sensor subsystem 412. The illustrated blocks represent structural and functional components that may be implemented in hardware, firmware, or combinations thereof. In certain embodiments, safety-critical components are implemented in physically separate hardware circuits to ensure independent enforcement of airflow safety constraints.

The wearable support structure 402 is configured to be worn on a user's head or face. In various embodiments, the wearable support structure 402 may include an eyewear-mounted frame, headband, helmet-style apparatus, or other head-supported structure. The wearable support structure 402 provides mechanical support and positional alignment for the nostril actuation assembly (Asymmetric & Ramp-controlled) 406, the sensor subsystem 412, and associated airflow pathways. Structural features may include rigid or semi-rigid support arms, adjustable nasal-contact mounts, and alignment guides configured to maintain consistent positioning relative to the user's nostrils. As illustrated, the wearable support structure 402 is operatively coupled to the mode selection interface 404 and the nostril actuation assembly (Asymmetric & Ramp-controlled) 406.

The mode selection interface 404 is configured to receive user input for selecting among a plurality of breathing exercise modes. As illustrated, the mode selection interface 404 may include: a manual input device (e.g., button, switch, knob, rotary selector); and/or non-mechanical input modalities (e.g., touch, tap, gesture, voice input). The mode selection interface 404 provides selected mode information and control inputs to the hardware safety supervisor module (independent safety Enforcement) 408 for further processing and safety validation.

The nostril actuation assembly (Asymmetric & Ramp-controlled) 406 is configured to selectively modulate airflow associated with at least one nostril of the user. In the illustrated embodiment, the nostril actuation assembly (Asymmetric & Ramp-controlled) 406 supports asymmetric operation, allowing different occlusion parameters for left and right nostrils; and ramp-controlled actuation, allowing gradual increases or decreases in occlusion force or airflow resistance to avoid abrupt physiological transitions. The nostril actuation assembly (Asymmetric & Ramp-controlled) 406 may include one or more actuators such as miniature motors, linear actuators, servo mechanisms, cam-driven elements, or magnetically actuated displacement members configured to position occlusion elements relative to the nostrils. The nostril actuation assembly (Asymmetric & Ramp-controlled) 406 does not receive unrestricted actuation authority from the control processor. Instead, actuation authority is logically and electrically gated by the hardware safety supervisor module (independent safety Enforcement) 408, as described below. The nostril actuation assembly (Asymmetric & Ramp-controlled) 406 is also coupled to the sensor subsystem 412 for receiving physiological and positional feedback.

The hardware safety supervisor module (independent safety Enforcement) 408 is configured to independently enforce predefined physiological and mechanical safety limits. Unlike a passive monitoring circuit, the hardware safety supervisor module (independent safety Enforcement) 408 is structurally positioned in the actuator command path and logically gates actuation authority. In certain embodiments, actuator drive enable signals must pass through hardware gating components controlled by the hardware safety supervisor module (independent safety Enforcement) 408 before reaching motor drivers associated with the nostril actuation assembly (Asymmetric & Ramp-controlled) 406. Accordingly, the hardware safety supervisor module (independent safety Enforcement) 408 may: permit actuation only when safety conditions are satisfied; inhibit actuator energization by de-asserting driver-enable lines; interrupt motor drive signals via hardware gating transistors or relays; and override control commands that would exceed predefined safety thresholds.

In certain embodiments, the hardware safety supervisor module (independent safety Enforcement) 408 may comprise: a physically separate microcontroller configured to independently validate actuation commands; a watchdog timing circuit configured to disable actuator drive upon exceeding maximum occlusion duration; FPGA-based state machine logic configured to deterministically gate actuator enable signals; or analog comparator circuitry configured to directly compare sensed pressure or force signals against predefined voltage thresholds and physically interrupt actuator drive circuitry. The hardware safety supervisor module (independent safety Enforcement) 408 may enforce limits including: maximum occlusion force, maximum continuous nostril closure duration, minimum airflow allowance, respiratory rate limits, and maximum breath-retention duration. The hardware safety supervisor module (independent safety Enforcement) 408 is further coupled to the passive mechanical airflow bypass path 410 as part of a layered safety architecture.

The passive mechanical airflow bypass path 410 provides a mechanically responsive airflow pathway configured to permit airflow independently of electronic control or powered actuation. To avoid purely functional interpretation, in certain embodiments the passive mechanical airflow bypass path 410 may comprise: a spring-biased flap valve positioned within an airflow channel and configured to open automatically when differential pressure across the valve exceeds a predefined spring force threshold; a pressure-relief diaphragm formed from an elastomeric membrane configured to deform and create an auxiliary airflow aperture when internal nasal pressure exceeds a predetermined limit; a pressure-actuated sliding gate biased toward an open position in the absence of actuator force; or a mechanically latched occlusion element configured to default to a non-restrictive position upon loss of electrical power.

The passive mechanical airflow bypass path 410 operates independently of electronic control signals and remains functional during power interruption, actuator malfunction, or safety-triggered shutdown. In this manner, airflow is mechanically restored if resistance exceeds a predefined threshold.

The sensor subsystem 412 includes one or more sensing elements configured to monitor system and physiological parameters. As illustrated, the sensor subsystem 412 may include: respiration sensors (e.g., airflow sensors, pressure sensors, acoustic sensors), fit-detection sensors positioned near nasal-contact regions, and redundant sensors for fault detection. The sensor subsystem 412 provides real-time feedback to both the nostril actuation assembly (Asymmetric & Ramp-controlled) 406 and the hardware safety supervisor module (independent safety Enforcement) 408. Sensor signals may be used to detect misalignment, displacement, abnormal respiratory conditions, excessive occlusion force, or inconsistent sensor readings.

In operation, the user interacts with the mode selection interface 404 to select a breathing exercise mode. The selected mode information is processed by the hardware safety supervisor module (independent safety Enforcement) 408, which evaluates the requested actuation parameters against predefined safety limits before enabling actuation. If safety conditions are satisfied, the hardware safety supervisor module (independent safety Enforcement) 408 asserts actuator-enable authorization, thereby permitting controlled operation of the nostril actuation assembly (Asymmetric & Ramp-controlled) 406. If safety limits are violated, the hardware safety supervisor module (independent safety Enforcement) 408 removes actuation authority by disabling actuator drive circuitry. During airflow modulation, the nostril actuation assembly (Asymmetric & Ramp-controlled) 406 modulates airflow in accordance with asymmetric and ramp-controlled parameters. The sensor subsystem 412 continuously monitors respiration and device alignment. If abnormal conditions are detected, the hardware safety supervisor module (independent safety Enforcement) 408 may immediately inhibit actuation and transition the system to a fully open airflow state. Independently of electronic control, the passive mechanical airflow bypass path 410 provides a structural fail-safe airflow route through, for example, a spring-biased flap valve or pressure-relief diaphragm. Thus, even if electronic actuation remains engaged or power is lost, airflow may be restored mechanically when pressure exceeds a safe threshold.

FIG. 4 therefore illustrates a layered safety architecture in which user input is validated before actuation is authorized, actuation authority is hardware-gated rather than merely monitored, physiological sensing supports real-time safety enforcement, and a passive mechanical airflow bypass path 410 provides power-independent fail-safe airflow. This architecture integrates mechanical, electrical, and logical safeguards to ensure that airflow modulation occurs only within predefined safety constraints while preventing complete airflow obstruction under abnormal conditions.

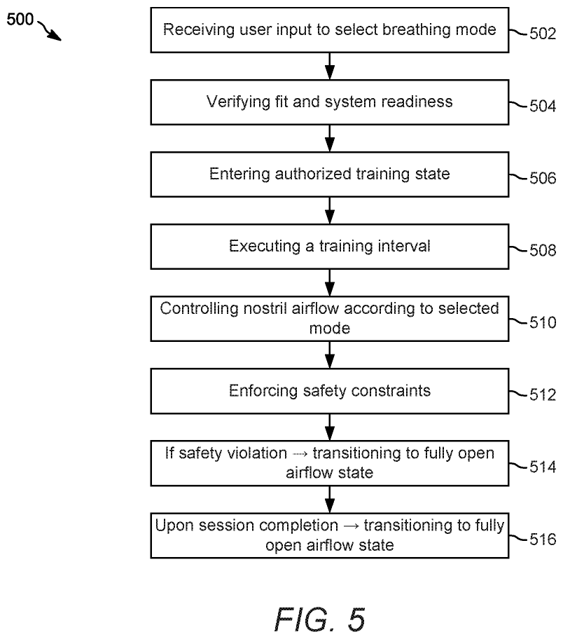

FIG. 5 illustrates an example method 500 for operating a wearable breathing system. The method 500 enables controlled nostril airflow modulation during structured breathing exercises while maintaining hardware-enforced safety constraints and automatic reversion to a fully open airflow state under predefined conditions.

The method 500 may be performed by a wearable breathing system including a wearable support structure configured to be worn on a user's head or face, a user-operable mode selection interface, a nostril actuation assembly (Asymmetric & Ramp-controlled) 406 configured to selectively restrict airflow through at least one nostril, a control system including at least one processor, a hardware-implemented safety supervisor module, respiration sensing elements, and associated airflow pathways including a passive mechanical airflow bypass path.

Although the operations are illustrated in a particular sequential order, the operations may be performed in a different order, in parallel, iteratively, or with certain steps omitted or combined depending on system implementation.

At block 502, the method includes receiving user input to select a breathing mode. The user input may be received via a user-operable mode selection interface including at least one manually actuated input device or at least one non-mechanical input modality, such as touch, tap, gesture, or voice input. In some embodiments, user input may be received via an external mobile application communicatively coupled to the wearable breathing system. The selected breathing mode may define one or more training intervals, timing parameters, occlusion force limits, ramp characteristics, inhalation duration, exhalation duration, nostril switching intervals, airflow restriction levels, breath-retention intervals, or combinations thereof.

At block 504, the method includes verifying fit and system readiness. Fit verification may include evaluating signals from fit-detection sensors positioned near nasal-contact regions to confirm proper actuator alignment relative to the user's nostrils. System readiness verification may include confirming actuator functionality, sensor integrity, available power levels, and safety supervisor operability prior to enabling airflow modulation. Passive monitoring of respiration sensing elements may occur during this stage while actuator drive circuitry remains disabled.

At block 506, the method includes entering an authorized training state. The authorized training state is entered only after successful verification of fit and system readiness. In certain embodiments, actuator drive circuitry is enabled through dual-channel gating logic requiring concurrent authorization from both a primary control processor and a hardware-implemented safety supervisor module (independent safety Enforcement) 408. Outside the authorized training state, airflow actuation components are maintained in a fully open airflow state.

At block 508, the method includes executing at least one training interval corresponding to the selected breathing exercise mode. A training interval may correspond to inhalation, exhalation, breath retention, or a composite breathing phase. Each training interval may include predefined duration parameters, ramp-up and ramp-down characteristics, and occlusion force profiles.

In certain embodiments, prior to or during execution of the training interval, a stepwise calibration routine may be performed to personalize occlusion parameters. The calibration routine may include: applying an initial low occlusion force to a nostril using the nostril actuation assembly, incrementally increasing occlusion force or actuator displacement in predefined steps, sampling airflow and/or pressure at each increment using respiration sensing elements, determining airflow reduction relative to a baseline airflow measurement, evaluating a convergence threshold defined as a percentage airflow reduction, stabilization over multiple sampling intervals, or achievement of a target resistance value, and recording the actuator position and corresponding force as an individualized minimum effective occlusion force. Calibration may be performed independently for left and right nostrils.

At block 510, the method includes controlling nostril airflow according to selected mode. Airflow control may include selective occlusion of one nostril while permitting airflow through the other nostril, asymmetric occlusion profiles, or bilateral airflow modulation. Controlled ramping may be implemented through servo positioning, linear actuator displacement, motor pulse-width modulation, or closed-loop force control based on pressure feedback.

At block 512, the method includes enforcing predefined physiological and mechanical safety limits using a hardware-implemented safety supervisor module (independent safety Enforcement) 408. In certain structural embodiments, the hardware-implemented safety supervisor module (independent safety Enforcement) 408 is physically separate from a primary control processor and may comprise: a dedicated secondary microcontroller configured to independently validate actuation commands; a watchdog timing circuit configured to monitor occlusion duration and disable actuator drive signals upon abnormal timing; a field-programmable gate array (FPGA) configured to perform deterministic state-based gating of actuator enable signals; or analog comparator circuitry configured to compare sensed pressure, airflow, or occlusion force signals against predefined voltage thresholds and to physically interrupt actuator drive lines when thresholds are exceeded.

In some embodiments, the hardware safety supervisor module (independent safety Enforcement) 408 is electrically interposed between the control processor and motor driver circuitry such that actuator enable signals must pass through hardware gating components before energizing the nostril actuation assembly. The hardware safety supervisor module (independent safety Enforcement) 408 may monitor maximum occlusion force, maximum continuous nostril closure duration, minimum airflow allowance, respiratory rate thresholds, or breath-retention duration limits.

At block 514, the method includes detecting a safety violation and transitioning to a fully open airflow state. If a predefined safety limit is exceeded, the hardware-implemented safety supervisor module overrides actuation commands and disables actuator drive signals. The nostril occlusion mechanisms are released so that airflow through one or both nostrils is substantially unrestricted by active actuation components. The transition to the fully open airflow state may include mechanical release of actuators, electrical de-energization of drive circuits, and default positioning of occlusion mechanisms to a non-restrictive configuration.

In certain embodiments, a passive mechanical airflow bypass path 410 provides additional fail-safe airflow. The passive mechanical airflow bypass path 410 may include: a spring-biased flap valve configured to open automatically when differential pressure exceeds a predetermined spring force threshold; or a pressure-relief diaphragm formed from an elastomeric membrane configured to deform and create an auxiliary airflow aperture when internal nasal pressure exceeds a predefined limit. Because the passive mechanical airflow bypass path operates independently of electronic control or power availability, airflow may be restored even in the event of power interruption or actuator malfunction.

At block 516, the method includes upon session completion, transitioning to the fully open airflow state. After completion of the final training interval, the system de-energizes actuator drive circuitry and repositions occlusion mechanisms to a non-restrictive configuration. The system may require renewed user input via the mode selection interface before re-entering the authorized training state.

Taken together, the method 500 provides a structured and safety-controlled approach to facilitating nostril breathing exercises. By integrating user input reception (502), readiness verification (504), authorized state control (506), training interval execution (508), airflow modulation (510), hardware-level safety enforcement (512), automatic fail-safe transition (514), and session-based reversion to a fully open airflow state (516), the wearable breathing system enables structured, repeatable, and hands-free nostril airflow modulation while maintaining independent mechanical and electronic safety protections.

The following examples illustrate operation of a wearable breathing system comprising a wearable support structure configured to be worn on a user's head or face and a user-operable mode selection interface coupled to the wearable support structure, wherein the mode selection interface is configured to enable selection between a plurality of breathing exercise modes for facilitating nostril breathing. These examples are non-limiting.

### Example 1: Selection of a Breathing Exercise Mode Via a User-Operable Mode Selection Interface

In one embodiment, a user wears a wearable breathing system comprising a wearable support structure configured to be worn on the user's head or face. The wearable support structure supports a nostril actuation assembly configured to control airflow associated with at least one nostril of the user. The system further includes a user-operable mode selection interface coupled to the wearable support structure. The mode selection interface 404 comprises at least one manually actuated input device configured to receive mechanical user input and/or at least one input device configured to receive user input via one or more non-mechanical input modalities comprising touch, tap, gesture, or voice.

The user provides input via the user-operable mode selection interface to select one breathing exercise mode from a plurality of breathing exercise modes. In this example, the selected breathing exercise mode defines a four-second inhalation interval, a four-second exhalation interval, and an alternating nostril sequence. In response to receiving the user input, the wearable breathing system selects the breathing exercise mode and controls airflow associated with at least one nostril of the user in accordance with the selected breathing exercise mode. The system performs airflow control without requiring manual nostril occlusion by the user.

### Example 2: Extended Breathing Session Without Manual Nostril Occlusion

In another embodiment, a user wears a wearable breathing system comprising a wearable support structure configured to be worn on the user's head or face and a user-operable mode selection interface coupled thereto.

The user selects, via the user-operable mode selection interface, a breathing exercise mode from a plurality of breathing exercise modes. The selected breathing exercise mode defines airflow restriction timing and duration parameters for at least one nostril. Upon selection, the wearable breathing system controls airflow associated with at least one nostril of the user in accordance with the selected breathing exercise mode. The system performs the airflow control for an extended duration, such as a thirty-minute breathing session.

Throughout the session, airflow is controlled automatically by the wearable breathing system without requiring manual nostril occlusion by the user. The user's hands remain free, and nostril airflow modulation is maintained consistently according to the selected breathing exercise mode.

### Example 3: Operation Using Non-Mechanical Input Modalities in an Immersive Environment

In a further embodiment, the wearable breathing system comprises a user-operable mode selection interface including at least one input device configured to receive user input via one or more non-mechanical input modalities comprising at least one of touch, tap, gesture, or voice.

A user wearing the wearable support structure on the head or face provides voice input to the mode selection interface to select a breathing exercise mode from a plurality of breathing exercise modes. In response to receiving the non-mechanical user input, the wearable breathing system selects the breathing exercise mode and controls airflow associated with at least one nostril in accordance with the selected breathing exercise mode.

In one implementation, the wearable breathing system is integrated with or worn concurrently with a virtual reality headset. The user remains engaged in an immersive environment while the wearable breathing system controls nostril airflow automatically and without requiring manual nostril occlusion by the user.

The following embodiments provide additional structural and operational examples of the wearable breathing system described herein. These embodiments are presented to illustrate representative implementations and are not intended to limit the scope of the invention. Unless otherwise indicated, components described in this section may be combined with any embodiments described elsewhere in the specification.

In certain embodiments, the airflow actuation assembly is configured to modulate airflow through one or both nostrils by varying an effective airflow aperture size or airflow impedance between multiple intermediate restriction levels between a fully open airflow state and a fully restricted state. Unlike binary occlusion devices, the airflow actuation assembly may permit graded or continuous modulation of airflow restriction. Such modulation enables the system to produce airflow profiles corresponding to inhalation phases, exhalation phases, breath retention phases, or composite breathing cycles.

Airflow modulation may be achieved through adjustment of one or more airflow control structures, including for example: a variable aperture, an iris mechanism, a compliant flow channel, a movable valve element, a sliding shutter, or a variable impedance element. These elements may be repositioned by controlled mechanical displacement produced by an actuator to produce partial airflow restriction corresponding to intermediate airflow states.

In certain embodiments, the airflow actuation assembly includes a mechanical biasing element configured to urge the airflow control mechanism toward a non-restrictive position. Example biasing mechanisms may include compression springs, torsion springs, elastomeric restoring elements, magnetic repulsion biasing, or gravity-assisted return configurations.

Electrical energy is therefore required to maintain airflow restriction. When electrical power is removed, the biasing element automatically returns the airflow control element toward the fully open airflow state. In some implementations, upon interruption of electrical power the airflow actuation assembly mechanically returns to the fully open airflow state within a predefined time interval, such as less than one second or another safety-defined interval.

In certain embodiments, the airflow actuation assembly includes mechanical stops or travel limiters configured to limit maximum occlusion displacement independently of electronic control signals. The mechanical stop may be implemented as: a rigid structural feature, an interference geometry, a displacement limiter, or a mechanical travel stop. Such structures physically prevent complete closure beyond a predefined safe displacement. Additionally, airflow restriction components may be spring biased toward a non-restrictive configuration, ensuring that restriction cannot persist in the absence of actuation energy.

In certain embodiments, system operation is governed by a deterministic operational state machine implemented by the control system and the hardware-implemented safety supervisor module (independent safety Enforcement) 408. The state machine may include operational states such as an idle state, a fit-verification state, an authorized training state, a fault state, and a lockout state.

Each state may correspond to a defined set of permissible system actions and actuator permissions. For example, in the idle state, the system remains inactive and airflow through the nostrils remains substantially unrestricted. In the fit-verification state, the system evaluates whether nasal-contact elements are properly positioned relative to the user's nostrils. Fit verification may include evaluation of signals from fit-detection sensors, airflow sensors, pressure sensors, or combinations thereof. The system enters the authorized training state only after successful completion of fit verification and confirmation of user intent. During this state, airflow modulation may be applied according to the selected breathing exercise mode. The system may transition to the fault state upon detection of abnormal operating conditions such as sensor disagreement, actuator malfunction, excessive occlusion force, or other safety violations. In certain implementations, the system may enter a lockout state following a safety violation. In the lockout state, airflow modulation is disabled until corrective actions are performed.

In some embodiments, transition from the fault state to the authorized training state requires renewed user input together with re-verification of device fit and sensor integrity. This deterministic state machine architecture allows predictable enforcement of safety rules during operation.

In some embodiments, the control system regulates airflow restriction using closed-loop feedback obtained from one or more sensing elements. Sensors suitable for closed-loop control may include airflow sensors, pressure sensors, force sensors associated with nasal contact surfaces, acoustic sensors configured to detect respiratory sounds, thermal airflow sensors, or combinations thereof. The control system may process sensor signals to maintain a target airflow, pressure, or resistance profile associated with a selected breathing exercise mode.

Closed-loop feedback may also enable the system to compensate for dynamic conditions occurring during use. For example, the system may detect nasal leakage, partial device misalignment, user head movement, or changes in respiratory effort. When such conditions are detected, the control system may adjust actuator displacement or airflow impedance to maintain the intended airflow restriction profile.

In certain embodiments, the system includes a fault-detection engine configured to identify inconsistencies between signals obtained from multiple respiratory sensing modalities. For example, the system may compare: the temporal phase correlation between two respiratory signals, or the magnitude coherence between respiratory signal amplitudes. Representative signal pairs may include airflow sensor signals compared with acoustic respiration signals, pressure signals compared with airflow signals, or airflow signals compared with nasal contact force signals. If the disagreement between two sensing modalities exceeds a predefined threshold, the system may determine that a sensing fault has occurred. Upon detection of such a sensing inconsistency, the system may disable actuator drive circuitry and transition the airflow actuation assembly to the fully open airflow state.

In certain embodiments, actuator enablement is governed by a dual-channel authorization architecture. In this architecture, actuator drive circuitry is enabled only when both of the following conditions are satisfied: i) a primary control processor issues an actuator enable command, and ii) a hardware-implemented safety supervisor module independently authorizes the command. The safety supervisor module may therefore act as a logical gate controlling actuation authority, rather than merely monitoring system behaviour. This dual-channel configuration reduces the likelihood that software faults or unintended commands could produce unsafe airflow restriction.

In some embodiments, the wearable support structure includes one or more skin-contact elements configured to interface with the user's face or nasal region. The skin-contact elements may be formed from medical-grade elastomeric materials, including for example: silicone, thermoplastic elastomer (TPE), thermoplastic polyurethane (TPU), hydrogel materials, or combinations thereof.

In certain embodiments, the skin-contact elements may incorporate antimicrobial additives or antimicrobial surface coatings configured to reduce microbial growth during repeated device use. In some implementations, nasal-contact elements may be configured as replaceable hygienic components. These elements may be formed from biodegradable or compostable materials and may be intended for single-user disposable replacement.

The airflow actuation assembly may employ various actuator mechanisms configured to modulate airflow restriction. Representative actuator types include cam-driven mechanical linkages, linear actuators, servo mechanisms, solenoids, magnetic displacement actuators, shape-memory alloy actuators, or inflatable bladder mechanisms. In certain embodiments, an inflatable bladder may be positioned adjacent to a nasal passage. The bladder may be pneumatically or electro-pneumatically actuated to produce controlled displacement of a nasal-contact surface, thereby modulating airflow restriction.

In addition to electronically controlled safety mechanisms, the system may include purely mechanical airflow safeguards. For example, the passive mechanical airflow bypass path may incorporate structural elements such as a spring-biased flap valve, a pressure-relief diaphragm, or a flexible membrane valve. These elements may automatically open when airflow resistance or pressure differential exceeds a predefined threshold. In some embodiments, the bypass path may further include a mechanical stop configured to limit maximum occlusion displacement independently of electronic control signals. This structure may prevent complete obstruction of airflow even if the actuation system were to malfunction.

In certain embodiments, the system may generate and present user-readable explanations describing automatically adjusted breathing parameters. Such explanations may be presented through a device display, a mobile application, an audio interface, or other user interface components. The explanations may identify triggering conditions responsible for system adjustments. Example triggering conditions may include airflow instability, excessive occlusion force, abnormal respiratory rate, sensor inconsistency, or fit misalignment. Providing such feedback may improve user understanding of system behaviour and promote safe device operation.

In some embodiments, the system permits user adjustment of certain breathing parameters while maintaining non-modifiable safety limits. These safety limits may include physiological limits and mechanical limits designed to prevent unsafe airflow restriction. Additionally, the system may detect a low battery condition and disable entry into the authorized training state until battery charge exceeds a predefined threshold level.

Methods of operating the wearable breathing system may include implementing a deterministic operational state machine governing transitions between idle, fit-verification, authorized training, fault, and lockout states. Upon detection of a safety violation, the method may include entering the lockout state and requiring renewed user confirmation together with re-verification of device fit before airflow modulation may resume.

In some embodiments, one or more non-transitory computer-readable media may store instructions that, when executed by one or more processors of the wearable breathing system, cause the system to perform operations including: verifying nasal-contact fit, entering an authorized training state only after validation by a hardware-implemented safety supervisor module, generating a timing profile for a selected breathing exercise mode, regulating airflow using closed-loop feedback, detecting safety violations or sensing inconsistencies, and transitioning to a fully open airflow state upon detection of a predefined safety condition.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

It is to be understood that the above description is intended to be illustrative, and not restrictive. For example, the above-discussed embodiments may be used in combination with each other. Many other embodiments will be apparent to those of skill in the art upon reviewing the above description.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions, and improvements to the embodiments described above are possible. It is contemplated that these variations, modifications, additions, and improvements fall within the scope of the invention.

We claim:

1. A wearable breathing system comprising:
a wearable support structure configured to be worn on a user's head or face;
airflow actuation components supported by the wearable support structure and configured to selectively restrict airflow through at least one nostril of the user without requiring manual nostril occlusion by the user; and
a user-operable mode selection interface coupled to the wearable support structure and including at least one input device configured to receive user input via one or more input modalities including a mechanical input modality or a non-mechanical input modality, wherein the mode selection interface is configured to enable user selection among a plurality of breathing exercise modes for facilitating nostril breathing, each breathing exercise mode defining airflow restriction timing and control parameters for the airflow actuation components.

2. The wearable breathing system of claim 1, wherein the mechanical input modality comprises a manually actuated input device and the non-mechanical input modality comprises at least one of touch input, tap input, gesture input, or voice input.

3. The wearable breathing system of claim 1, further comprising a hardware-implemented safety supervisor module electrically interposed between a control processor and the airflow actuation components and configured to enforce predefined physiological and mechanical safety limits independently of user commands, mobile applications, or cloud software, and to override actuation commands that would violate the safety limits.

4. The wearable breathing system of claim 3, wherein the safety limits include at least one of a maximum nostril occlusion force, a maximum continuous nostril closure duration, a minimum airflow allowance, a minimum respiratory rate, a maximum respiratory rate, or a maximum breath-retention duration.

5. The wearable breathing system of claim 1, wherein the wearable breathing system includes a passive mechanical airflow bypass path comprising at least one of a spring-biased flap valve, a pressure-relief elastomeric diaphragm, or a pressure-actuated sliding gate, configured to automatically open when airflow resistance exceeds a predefined threshold, independent of electronic actuation or power availability.

6. The wearable breathing system of claim 1, wherein the system includes redundant or heterogeneous respiration sensing elements and a fault-detection engine configured to detect sensor malfunction, misalignment, or displacement and to revert the system to a fully open airflow state upon detection of sensing inconsistency.

7. The wearable breathing system of claim 1, wherein the wearable breathing system further comprises a personalization and calibration module to perform an automatic nostril calibration routine to establish an individualized minimum effective occlusion force and actuator geometry based on nasal compliance and airflow response.

8. The wearable breathing system of claim 1, wherein the wearable breathing system includes a self-aligning nostril interface configured to dynamically adjust actuator positioning to maintain consistent alignment during user movement or extended wear.

9. The wearable breathing system of claim 1, wherein the system supports asymmetric nostril control profiles in which occlusion timing, occlusion force, and airflow resistance differ independently between left and right nostrils.

10. The wearable breathing system of claim 1, wherein the wearable breathing system includes a modular actuator interface having standardized mechanical and electrical connectors configured to interchangeably receive multiple nostril-actuation modules, each module being automatically identified to apply corresponding control limits and calibration parameters.

11. The wearable breathing system of claim 1, wherein the system comprises disposable or limited-use nasal-contact elements tracked by a usage counter and configured to prompt replacement after a predetermined usage threshold.

12. The wearable breathing system of claim 1, wherein the system includes fit-detection sensors positioned near a nasal-contact region to assess contact quality and alignment and to provide corrective guidance prior to initiating a breathing session.

13. The wearable breathing system of claim 1, wherein the system calculates at least one session metric score and presents the score as an indication of breathing session effectiveness.

14. A method of operating a wearable breathing system worn on a user's head or face, the method comprising:
receiving user input via a user-operable mode selection interface of the wearable breathing system;
selecting a breathing exercise mode based on the user input; and
controlling airflow associated with at least one nostril of the user in accordance with the selected breathing exercise mode by actuating one or more airflow actuation components of the wearable breathing system to selectively restrict nostril airflow, wherein the method is performed by the wearable breathing system without requiring manual nostril occlusion by the user.

15. The method of claim 14, further comprising enforcing predefined physiological and mechanical safety limits using a hardware-implemented safety supervisor module operating independently of user commands, mobile applications, or cloud software, and overriding airflow control actions that would violate the safety limits.

16. The method of claim 14, further comprising automatically reverting the wearable breathing system to a fully open airflow state in response to at least one of (i) airflow resistance exceeding a predefined threshold or (ii) detection of malfunction, misalignment, or inconsistency among respiration sensing elements.

17. The method of claim 14, further comprising implementing a deterministic operational state machine governing transitions among a plurality of operational states including at least an idle state in which airflow actuation is disabled, a fit-verification state in which nasal-contact fit and sensor integrity are confirmed prior to enabling actuation, an authorized training state in which airflow modulation is permitted in accordance with the selected breathing exercise mode, and a fault state in which actuation is inhibited upon detection of a safety violation or sensing inconsistency, wherein transition from the fault state to the authorized training state requires renewed user confirmation together with re-verification of device fit.

18. The method of claim 14, further comprising enforcing gradual ramp-up and ramp-down phases when applying or releasing airflow restriction to prevent abrupt physiological transitions.

19. A wearable breathing apparatus, comprising:
a wearable support structure configured to be worn on a user's head or face;
an airflow actuation assembly supported by the wearable support structure and configured to selectively modulate airflow associated with at least one nostril of the user;
at least one respiration sensing element configured to detect at least one respiratory parameter of the user;
a control system operatively coupled to the airflow actuation assembly and the at least one respiration sensing element; and
a hardware-implemented safety supervisor module operatively coupled between the control system and the airflow actuation assembly,
wherein the hardware-implemented safety supervisor module is configured to:
(i) determine an operational state of the wearable breathing apparatus based on at least one of sensed respiratory parameters, airflow resistance, actuation force, elapsed occlusion duration, or system fault conditions;
(ii) enforce predefined physiological and mechanical safety limits corresponding to the determined operational state; and
(iii) gate, modify, or override actuation commands issued by the control system to prevent airflow restriction conditions that exceed the predefined safety limits, wherein the airflow actuation assembly is permitted to modulate airflow only when both the control system and the hardware-implemented safety supervisor module independently assert concurrent authorization, such that actuation is inhibited if either channel fails to assert an enable condition.

20. The wearable breathing apparatus of claim 19, wherein the hardware-implemented safety supervisor module comprises at least one of:
a dedicated microcontroller physically separate from the control system and configured to independently evaluate actuation commands prior to permitting operation of the airflow actuation assembly;
a hardware watchdog circuit configured to monitor an operational timing parameter associated with airflow restriction and to disable actuator drive signals upon detection of an abnormal timing condition;
a field-programmable gate array configured to implement deterministic state-based gating logic that conditionally permits or blocks actuator control signals; or analog comparator circuitry configured to compare sensed pressure, airflow, or occlusion force signals against predefined threshold values and to interrupt actuator drive signals when a threshold condition is exceeded.
